(19) United States
(12) Patent Application Publication
(10) Pub. No.: US 2025/0064676 A1
(43) Pub. Date: Feb. 27, 2025

(54) AN AMPOULE

(71) Applicant: BEYNON BOYLE MEDICAL LTD, Teddington, Middlesex (GB)

(72) Inventor: Jeremy Boyle, Teddington, Middlesex (GB)

(21) Appl. No.: 18/718,134

(22) PCT Filed: Dec. 8, 2022
(86) PCT No.: PCT/EP2022/085054

§ 371 Date: Jun. 10, 2024

(30) Foreign Application Priority Data
Dec. 10, 2021 (GB) 2117918.9

(51) Int. Cl. A61J 1/06

(52) U.S. Cl. CPC A61J 1/067

(57) ABSTRACT

The present invention relates to a drug delivery device and particularly, although not exclusively, to an ampoule which may be used to deliver drugs directly to a patient. In particular, the present invention provides an ampoule for delivering fluid directly to a patient, wherein the ampoule comprises; a body portion which is compressible to expel fluid from the ampoule, and a connector for fitting the ampoule to a cannula or a hollow needle to deliver fluid therethrough. In this way, the present invention provides an ampoule which may be connected directly to a cannula or a hollow needle to allow a clinician to deliver fluid to a patient (e.g. as an injection), reducing waste and risk associated with using syringes or the like.

### FIELD OF THE INVENTION

The present invention relates to a drug delivery device and particularly, although not exclusively, to an ampoule which may be used to deliver drugs directly to a patient.

### BACKGROUND

Before a clinician, such as a doctor or a nurse, delivers an injectable medicine to a patient several steps are required to prepare the treatment. This includes the use of a significant amount of apparatus, much or all of which then needs to be discarded as clinical waste (which is typically incinerated).

For example, drugs are typically stored within individual glass ampoules. In order to deliver the drug to a patient, a clinician must first prepare a syringe by connecting it to a hypodermic needle, use the syringe and needle to aspirate (draw up) the drug from the ampoule, and then deliver the drug to the patient, for example using the hypodermic needle or a separate cannula. After the drug has been delivered, the syringe (including the syringe barrel and the syringe plunger), hypodermic needle and ampoule must all be disposed of. Each of these components will typically have associated casing and packaging, which must also be disposed of. It will therefore be appreciated that this process generates a lot of waste, particularly when considering the volume of injectable medicaments which are routinely delivered to patients.

Furthermore, the steps involved with preparing the syringe and the drug for delivery to a patient may risk the health of the patient and/or of the clinician. For example, a mistake may be made in aspirating a drug from the ampoule and delivering the correct amount to the patient. The aspirating step also introduces a delay to drug delivery, which may be critically important in certain scenarios where the time taken to draw up the drug into a syringe can be the difference between a positive and a negative outcome for the patient. Additionally, handling needles presents a health and safety hazard, as steps which require a clinician to handle a hypodermic needle will inevitably increase the risk of a needle stick injury or the like.

The present invention has been devised in light of the above considerations.

### SUMMARY OF THE INVENTION

Broadly, the present invention provides an ampoule, which may be referred to herein as a primary drug container (PDC), which can be used to directly deliver a fluid to a patient. Preferably, the ampoule of the present invention is usable to deliver fluid intravenously (e.g., through a giving set, an IV line, a catheter, a cannula, or the like).

In particular, according to a first aspect of the present invention there is provided an ampoule (a primary drug container) for delivering fluid directly to a patient, wherein the ampoule comprises: a body portion which is compressible to expel fluid from the ampoule, and a connector for fitting the ampoule to a cannula (for example an intravenous (IV) access port, side port, a giving set, a catheter, or the like) or a hollow needle (such as a hypodermic needle, trocar needle, or the like) to deliver fluid therethrough. In this way, the first aspect of the present invention provides an ampoule which may be connected directly to a cannula or a hollow needle to allow a clinician to deliver fluid to a patient (e.g. as an injection, or preferably through an IV access port, side port, a giving set, a catheter or the like), without the steps associated with aspirating a drug from a typical ampoule into a syringe before the fluid can be delivered. As well as saving time in delivering the fluid, thereby increasing healthcare efficiency, by removing steps where human errors can be made from the process the process of fluid delivery is made safer. Furthermore, much less waste is generated, making the ampoule of the present invention more economical (as less associated apparatus is required) and environmentally friendly.

The current way to give drugs is by connecting a syringe (which has been used to aspirate the drugs from an ampoule) to a needle or a cannula. However, sometimes a bag of fluid is run into a patient via a tube called a ‘giving set’ which is connected to the cannula. This ‘giving set’ has access ports and/or side ports, which may be built into the giving set or connected into the system by a clinician. There are also extensions sometimes connected to the cannula which can also be injected into. By being configured as outlined above, an ampoule according to the present invention can be used to directly deliver fluid to a patient through any of these known components, thereby eliminating the need for a syringe or aspirating needle. This reduces the generation of waste and increases healthcare efficiency as discussed above.

Optionally, the body portion may be configured to maintain a compressed state after a compressing force has been applied. For example, the body portion may be configured to maintain a compressed state after it has been entirely compressed such that all of the fluid within the ampoule has been expelled, and/or it may be configured to maintain a partially compressed state when only a portion of the fluid within the ampoule has been expelled. The body portion may be configured in this way due to its shape, structure, and/or due to the materials from which it is manufactured. For example, the body portion may comprise a plastic or elastomeric material which is rigid enough to maintain a compressed or deformed state after pressure has been applied. That is, the body portion may preferably be made of a substantially inelastic material to achieve this effect. By being configured in this way, it is ensured that no fluid or air is drawn back into the ampoule after fluid has been expelled due to pressure exerted on the body portion a clinician.

Optionally, the ampoule may further comprise a valve, such as a check valve or a one way valve, to prevent fluid (e.g., the same fluid which is delivered, or a different fluid such as air) flow into the ampoule. For example, the connector may comprise a valve. This may ensure that fluid which is expelled from the ampoule cannot flow back into the ampoule, e.g., during fluid delivery. In certain embodiments, the valve may be positioned within a ‘neck’ portion of the ampoule, for example where a body portion and a connector of the ampoule meet. The valve may be made of the same material as any other part of the ampoule, or may be made of a different material; for example a plastic or an elastomeric material.

Optionally, the body portion may be preferentially compressible in one dimension. That is, it may be easier to compress the body portion in one plane or dimension than other planes or dimensions. For example, the body portion may be shaped to achieve this effect, and/or the manufacturing material may aid this effect. In certain embodiments, for example, the body portion may be generally tubular with an elliptical cross-section, such that the body portion is preferentially compressible in a dimension which is aligned with the minor axis of the elliptical cross-section and the body portion is resistant to compression in other dimensions. By being configured in this way, a clinician which is using the ampoule to deliver fluid directly to the patient may have greater control of the compression and fluid delivery, whilst also ensuring that the ampoule is rigid enough to resist deformation during transportation (e.g., particularly across the other axes).

Optionally, the body portion may comprise a rigid portion and a flexible portion. For example, the body portion may be compressible by applying pressure to a flexible portion, and the rigid portion may be provided to help hold and/or use the ampoule as well as strengthening the ampoule for easy packaging and/or transportation (in addition to, for example, reducing the risk of leakages). In some examples, pressure may be applied to a flexible portion by a user actuating or otherwise applying pressure to a rigid portion of the body. In other examples, a flexible portion may be pressed, or compressed, directly by a user. Generally, the rigid portion may provide structural support (e.g., in the form of a cuff or other feature), and the flexible portion may be pressable or otherwise movable in order to expel fluid from the body portion. In some examples, a flexible portion may provide a bladder or the like for containing the fluid to be delivered. It will be appreciated that, in embodiments, the body portion may comprise any suitable number of such rigid portions and/or flexible portions, and it is not required for the number of rigid portions to be equal to the number of flexible portions. For example, some embodiments may comprise two or more flexible portions.

By providing different arrangements of rigid portions and flexible portions, an ampoule according to the present invention may be provided in any suitable form, preferably which may be easily used to deliver fluid therefrom. In particular, it should be understood that the present invention is not bound by any particular geometry and/or relative arrangement of rigid portions and flexible portions.

It will be understood that, when used here, the terms ‘flexible’ and ‘rigid’ are relative terms, such that a portion which is described as ‘flexible’ has a greater flexibility and/or deformability than a portion which is described as ‘rigid’, and vice versa. For example, a ‘rigid’ portion may have a higher material strength and/or stiffness than a ‘flexible’ portion, and vice versa.

Optionally, the body portion may be made of a single material, such as a plastic material or an elastomeric material. For example, the one or more rigid portions and one or more flexible portions may be made of the same material. For example, a different thickness of the material may be used in different parts of the body portion. In some embodiments, the one or more rigid portions may comprise the same material with a different thickness from the one or more flexible portions to provide the required rigid or flexible properties for each respective portion, for example. For example, a rigid portion may have a material thickness of greater than 1 mm, such as 1.2 mm or 2 mm or more, whereas a flexible portion may have a material thickness of less than 1 mm, such as 0.5 mm or less.

In other examples, the body portion may be formed of a plurality of materials, such as plastic materials and/or elastomeric materials, wherein the choice of material itself provides the desired properties for each part. For example, the one or more rigid portions may comprise a different material from the one or more flexible portions. In some examples, a plurality of rigid portions may comprise the same material or may comprise different materials. In a like manner, in some examples, a plurality of flexible portions may comprise the same material or may comprise different materials.

In some examples, optionally, the ampoule may further comprise a circumferential ridge or cuff, providing a rigid portion, around the body portion (or a flexible portion, or bladder, of the body portion) for stiffening the body portion. As well as providing additional strength and rigidity to the ampoule, this may help to ensure that the body portion is preferentially compressible in one plane, and will resist compression in a plane generally aligned with the circumferential ridge. In certain embodiments, the circumferential ridge may be provided as a planar flange which extends outwardly from the ampoule. The planar flange may help to provide a gripping portion for the ampoule, which may be particularly advantageous where the ampoule is configured to hold and deliver a small amount of fluid (e.g. 5 ml or less) and so has a corresponding small size. Additionally or alternatively, the circumferential ridge may be formed of a different material to the body portion; though the circumferential ridge may in some embodiments be formed of the same material as the body portion. For example, the body portion may be made of or comprise a material, or a thickness of material, which is less rigid and easier to compress to aid fluid delivery, whereas the circumferential ridge may be made of or comprise a material, or a thickness of material, with a higher rigidity to provide structural strength for the ampoule.

Optionally, the ampoule may be formed from two sheets of material which are sealed together at their edges to define the body portion and an opening for delivering fluid therethrough. The ampoule of the present invention may thereby be cheap and simple to manufacture. For example, the material for forming the ampoule and/or the body portion may be a plastic material, and/or an elastomeric material preferably an inert plastic and/or elastomeric material to ensure that the ampoule does not react with the fluid contained therein. In some embodiments, the seal which is formed at the end of the ampoule may form the circumferential ridge referred to above.

In some embodiments, the ampoule may be formed through a blow-fill-seal (BFS) method. For example, the ampoule may be formed from a plastic and/or an elastomeric material which can be extruded and filled with fluid to be delivered to a patient. In some embodiments, the BFS method may form one or more rigid portions (e.g., a circumferential ridge or cuff) and one or more flexible portions of the body portion.

Optionally, the ampoule may taper towards a first end, and liquid may be expelled from the ampoule through the first end. This may help to ensure that the entire dose of fluid contained within the ampoule is delivered to a patient, which may be particularly advantageous in embodiments where the ampoule contains a single dose of fluid. In some embodiments, the ampoule may comprise a valve, such as a one-way valve or check valve, towards a first end of the ampoule in the tapered section.

Optionally, the ampoule may taper at a second, closed, end. For example, the ampoule may be rounded at a second end. This may aid control of an air bubble which may be present within the body portion in order to help a user to avoid accidentally delivering air with the fluid to be delivered (for example, by helping a user to ‘trap’ the air bubble at the tapered or rounded portion at the second end).

Optionally, the ampoule may comprise a tamper evident closure. For example, the closure may require a seal to be broken, or removed, in order to open and use the ampoule. This may help a user, in particular a clinician, ensure that the ampoule has not been opened prior to use, providing reassurance that the fluid is safe to be delivered to a patient. For example, the seal may comprise a frangible member, which may be located at or about the connector, which needs to be broken in order to use the ampoule. In another example, the tamper evident closure may comprise a seal (e.g., a foil seal) which needs to be removed from an opening before fluid can be delivered. In some embodiments, the tamper-evident closure may be a cap which is configured to enclose the connector. In this way, the cap may also ensure that the connector kept sterile until the ampoule is opened and the fluid is delivered via a hollow needle. In other examples, the ampoule may be sealed at the connector, and the ampoule may be opened by snapping a portion off from the connector. This seal portion may thereby form a tamper evident closure. Additionally or alternatively, the closure may be coloured to indicate the fluid contained in the ampoule, which may ensure a clinician is able to quickly determine and verify which fluid is within the ampoule to be delivered to a patient.

Optionally, the connector may be a luer connector. For example, the connector may be a luer slip (or slip tip) connector, in particular a luer male taper fitting. Alternatively, the connector may be a luer lock fitting. These connectors may give a simple and direct connection to a range of widely used medical instruments, allowing the ampoule to be used to directly deliver fluid to a patient in a range of circumstances without requiring the use of a syringe or the like.

Optionally, the ampoule may be configured to contain no more than about 20 ml of fluid. For example, the ampoule may be configured to contain 1 ml, 2 ml, 5 ml of fluid, 10 ml of fluid, or 15 ml of fluid. In this way the ampoule may be configured to contain and deliver a single dose, which may be delivered in one bolus.

Optionally, the ampoule may comprise markings for indicating the amount of fluid contained within the ampoule. This may aid a clinician in ensuring that they have selected an ampoule containing the correct dose of fluid, and may also be used by a clinician to assess how much fluid has been delivered to a patient. This may be particularly advantageous in embodiments where the ampoule contains multiple doses, or where less than a full dose is to be delivered to a patient, for example.

Optionally, the fluid is an injectable medicant, such as an anaesthetic, a saline (sodium chloride) solution, or an anti-sickness drug for example.

Optionally, the ampoule made be made of a single material. In other embodiments, the ampoule made be made of more than one material (e.g., the body portion may comprise one or more materials, and the connector may comprise one or more different materials). For example, the or each material may be a plastic material or an elastomeric material.

According to a second aspect of the present invention, there is provided an apparatus for delivering fluid to a patient, the apparatus comprising an ampoule according to the first aspect of the invention, and a cannula (for example an intravenous (IV) access port, side port, a giving set, a catheter, or the like). In this way, the second aspect of the present invention allows fluid to be delivered directly to a patient through a cannula, without the waste and other disadvantages associated with known methods (e.g. using a syringe or the like).

According to a third aspect of the present invention, there is provided an apparatus for delivering fluid to a patient, the apparatus comprising an ampoule according to the first aspect of the invention, and a hollow needle. In this way, the second aspect of the present invention allows fluid to be delivered directly to a patient through a hollow needle (such as a hypodermic needle), without the waste and other disadvantages associated with known methods (e.g. using a syringe or the like).

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a perspective view of an ampoule according to an embodiment of the present invention;

FIG. 2 shows a perspective view of a second ampoule according to a further embodiment of the present invention;

FIG. 3 shows a cross-sectional view of a third ampoule according to another embodiment of the present invention;

FIG. 4 shows a side view of a fourth ampoule according to another embodiment of the present invention;

FIG. 5 shows a cross-sectional view of a fifth ampoule according to another embodiment of the present invention;

FIG. 6 shows a top view of a sixth ampoule according to another embodiment of the present invention;

FIG. 7 shows a side view of the ampoule shown in FIG. 6;

FIG. 8 shows a perspective view of a first valve arrangement which may be used in embodiments of the present invention;

FIG. 9 shows a cross-sectional view of a second valve arrangement which may be used in embodiments of the present invention;

FIG. 10 shows a cross-sectional view of a third valve arrangement which may be used in embodiments of the present invention; and

FIG. 11 shows a cross-sectional view of a fourth valve arrangement which may be used in embodiments of the present invention.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

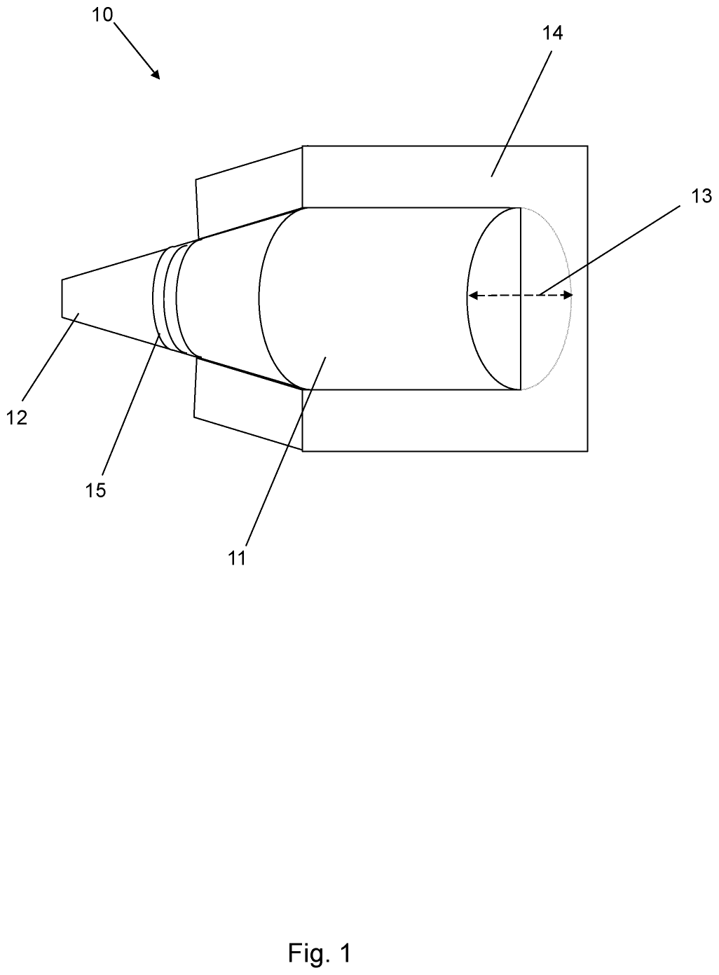

FIG. 1 shows a perspective view of an ampoule 10 according to an embodiment of the present invention. The ampoule 10 comprises a body portion 11 which is configured to contain fluid for delivery to a patient. For example, the fluid may be an injectable medicant, such as an anaesthetic, a saline (sodium chloride) solution, or an anti-sickness drug. The body portion 11 is compressible in order to expel the fluid. The ampoule 10 also comprises a connector 12 for fitting the ampoule 10 to a hollow needle to deliver the fluid therethrough. For example, the ampoule 10 may be connected to a hypodermic needle or a cannula in order to deliver fluid directly to a patient. The connector 12 shown in FIG. 1 is a male luer slip connector, though it will be appreciated that any other suitable type of connector may be contemplated. It will also be appreciated from FIG. 1 that the ampoule 10 tapers towards the connector 12, which helps to ensure that the entire dose of fluid contained within the ampoule 10 is delivered to a patient when a compressive force is applied to the body portion 11.

The body portion 11 is a tubular member, preferably made of plastic, which contains the fluid to be delivered to a patient. The cross-sectional shape of the body portion 11 is an ellipse. By being shaped in this way, the body portion 11 is relatively easy to compress along the minor axis of the elliptical cross-section, that is, in the direction shown by arrow 13. In other planes or dimensions the body portion 11 is more difficult to compress and so resists deformation when a force is applied. Such a configuration helps to ensure that the body portion 11 is rigid enough for transportation while allowing a user to readily control fluid delivery from the ampoule 10.

To further aid rigidity of the body portion 11, the ampoule 10 comprises a circumferential ridge in the form of a flange 14. The flange 14 stiffens the body portion 11 against bending and compressive deformation applied perpendicularly to the flange, while allowing the body portion 11 to be compressed along its minor axis shown by arrow 13. The flange 14 may also be useful to help a user to grip the ampoule 10, which may help them to compress the body portion 11 and expel fluid from the ampoule 10. In some examples, the flange 14 may be formed of material which is different from the rest of the ampoule 10, and in particular the material may be different from the body portion 11. For example, the flange 14 may be formed of a rigid material, while it may be preferable for the body portion 11 to be formed of a less rigid material which is easier for a user to compress. In other examples, the flange 14 may be formed from the same material as the body portion 11. For example, the ampoule 10 may be formed of two sheets of material which are shaped to form the two halves of the ampoule 10. The sheets may be joined at their edges to define the body portion 11 and an opening at the connector 12 to allow fluid to be delivered therethrough. The flange 14 may be formed from the parts of the two sheets which are joined to one another. For example, the ampoule 10 may be made of a plastic material which is heat formed, and the flange 14 is formed when the edges of the two sheets are sealed together with heat and pressure.

The elliptical cross-sectional shape of the body portion 11 in conjunction with the material it is made from may ensure that the body portion 11 is configured to maintain a compressed state after a compressing force has been applied by a user. For example, the body portion 11 may be made from a material having a low elasticity (in other words, a high plasticity) so that the body portion 11 tends to retain its shape after a force has been applied. The shape of the body portion 11 also contributes to this effect, by allowing rather than resisting deformation. In this way it can be ensured that no fluid is drawn back into the ampoule 10 after fluid has been expelled.

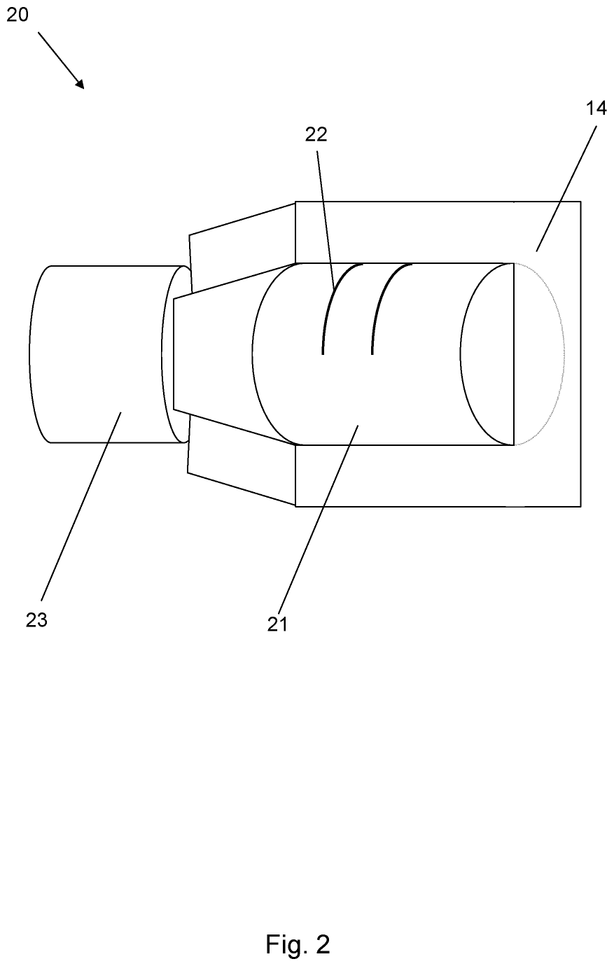

The ampoule 10 further comprises threads 15 configured to receive a screw cap which may be fitted over the connector 12. For example, the cap may be a tamper-evident cap which has a seal or the like to indicate to a user when the ampoule 10 has been opened. The cap may cover the connector 12 to ensure that the connector 12 remains sterile when the ampoule 10 is transported and until the ampoule 10 is fitted to a cannula or a hollow needle (for example, an intravenous (IV) access port, side port, a giving set, a catheter, or the like) to deliver fluid to a patient. The cap may be a cap as discussed below with respect to FIG. 2, for example.

It will be appreciated that, in certain embodiments, the ampoule 10 may be further provided with a valve, for example a valve as described below with respect to one of FIGS. 8-11.

FIG. 2 shows a perspective view of a second ampoule 20 which is a further embodiment of the present invention. Where features of the second ampoule 20 are the same as those described above with respect to the ampoule 10 shown in FIG. 1, a description of those features has not been repeated.

The ampoule 20 comprises a body portion 21 which is configured to contain fluid for delivery to a patient. In this embodiment, the ampoule 20 comprises markings 22 which indicate the amount of fluid contained within the ampoule 20. In particular, the markings 22 are volumetric markings on the body portion 21 which enable a user to see how much fluid is contained within the body portion 21. These markings may be useful to ensure that the ampoule 20 contains the correct amount of a particular fluid for a single dose, or may be used to assess how much liquid has been expelled from the ampoule 20 if not all of the fluid is intended to be delivered to a patient. For example, the markings 21 may indicate the amount of fluid within the body portion to the nearest millilitre, or 5 millilitres.

FIG. 2 shows the ampoule 20 with a cap 23 positioned so as to enclose the connector of the ampoule 20. In this way, the sterility of the connector may be maintained until the cap 23 is removed. The cap 23 may be a screw cap which is fitted to a thread or threads, such as threads 15 discussed above with respect to FIG. 1. The cap 23 is a tamper-evident cap to enable a user to determine whether the ampoule 20 has been opened prior to their use. For example, the base of the cap 23 may have a breakable connection with a ring or loop which is retained over the connector of the ampoule 20 when the cap 23 is removed. If the connection between the cap 23 and the loop is broken, then this may be taken as an indication that the ampoule 20 has been opened previously. Appropriate action may then be taken by a user, such as disposal of the ampoule 20, as the fluid within the ampoule 20 may no longer be suitable for delivery to a patient.

In some embodiments, the cap 23 may be coloured to indicate the fluid which is contained in the ampoule 20. For example, a first colour (e.g. yellow) may indicate a first medicament (such as an anaesthetic) is contained in the ampoule 20, and a second colour (e.g. beige) may indicate a second medicament (such as an anti-sickness drug) is contained in the ampoule 20.

To deliver fluid to a patient, the ampoule 20 is first opened by removing the cap 23. This exposes the connector portion, allowing the ampoule 20 to be connected directly to a cannula (for example an intravenous (IV) access port, side port, a giving set, a catheter, or the like) or to a hollow needle (such as a hypodermic needle). The clinician uses the cannula or needle to deliver fluid to a patient as normal, and expels fluid from the ampoule 20 by compressing the body portion 21. If the body portion 21 contains a large dose (e.g. 20 ml), the clinician may compress the body portion 21 at its distal end, furthest from the connector, and use the markings 22 as a guide for how much fluid has been delivered to the patient. In this way, the ampoule 20 is directly used to deliver fluid to a patient (in particular a medicament such as an anaesthetic), and so the wastage and risks associated with syringes can be avoided.

It will be appreciated that, in certain embodiments, the ampoule 20 may be further provided with a valve, for example a valve as described below with respect to one of FIGS. 8-11.

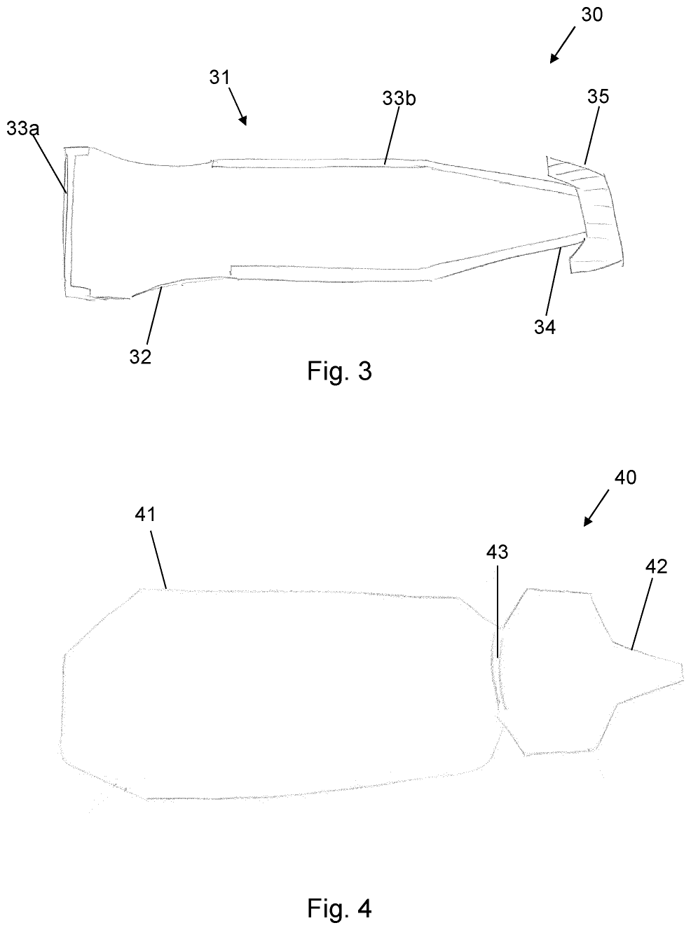

FIG. 3 shows a cross-sectional view of an ampoule 30 according to a further embodiment of the present invention.

The ampoule 30 comprises a body portion 31 and a connector 34 for fitting the ampoule 30 to a cannula or the like. The connector 34 may be a luer connector, such as a luer lock or luer slip connector, or any other suitable connector.

The body portion 31 is a generally tubular (e.g., cylindrical) arrangement which is configured to contain a fluid for delivery to a patient. As shown in FIG. 3, the body portion 31 tapers towards the connector 34 at a first end, where fluid is expelled from the ampoule 30. The body portion 31 comprises a flexible portion 32, which extends circumferentially around the body portion 31 perpendicular to its longitudinal axis, and two rigid portions 33a, 33b. In this way, the body portion 31 may be compressed to expel fluid by exerting pressure on the flexible portion 32. For example, a user may directly apply pressure to the flexible portion 32 (e.g., by squeezing or pressing the flexible portion 32), or a user may apply pressure to a first rigid portion 33a to move it towards a second rigid portion 33b, thereby compressing the flexible portion 32. The rigid portions 33a, 33b also provide structural strength and rigidity to the body portion 31, which may be useful, for example, in packing and transporting the ampoule 30.

The body portion 31 may be made of a single material, such as a plastic or an elastomer, wherein the flexible portion 32 and the rigid portions 33a, 33b may comprise different thicknesses of the material. However, it will be appreciated that in some examples, the body portion 31 may comprise any suitable number of different materials. For example, the flexible portion 32 may be made of a first material, and the rigid portions 33a, 33b may be made of a second material. Alternatively, the first rigid portion 33a may be made of a second material and the second rigid portion 33b may be made of a third material, for example. Optionally, the first, second and third materials may each be a plastic material or an elastomeric material, but it will be appreciated that other suitable materials may also be chosen.

The ampoule 30 comprises a tamper evident closure in the form of a snap-off cap 35, which must be removed from the connector 34 before the ampoule 30 can be used.

It will be appreciated that, in certain embodiments, the ampoule 30 may be further provided with a valve, for example a valve as described below with respect to one of FIGS. 8-11.

FIG. 4 shows a side view of another ampoule 40 according to a further embodiment of the present invention.

The ampoule 40 comprises a body portion 41 and a connector 42 for fitting the ampoule 40 to a cannula or the like. The connector 42 may be a luer connector, such as a luer lock or luer slip connector, or any other suitable connector

In this embodiment, the body portion 41 comprises a flexible material (for example, a plastic material or elastomeric material) which forms a bladder, and is thereby configured to contain fluid for delivery to a patient. In this way, when the body portion 41 contains fluid, it is compressible by a user in order to expel that fluid for delivery.

Between the body portion 41 and the connector 42, in a portion of the ampoule which may be referred to as the ‘neck’ portion, the ampoule 40 comprises a frangible member 43 which provides a tamper-evident closure for the ampoule 40. In order to deliver fluid, a user must first break the frangible member 43 (e.g., by compressing or squeezing the frangible member 43).

It will be appreciated that, in certain embodiments, the ampoule 40 may be further provided with a valve, for example a valve as described below with respect to one of FIGS. 8-11.

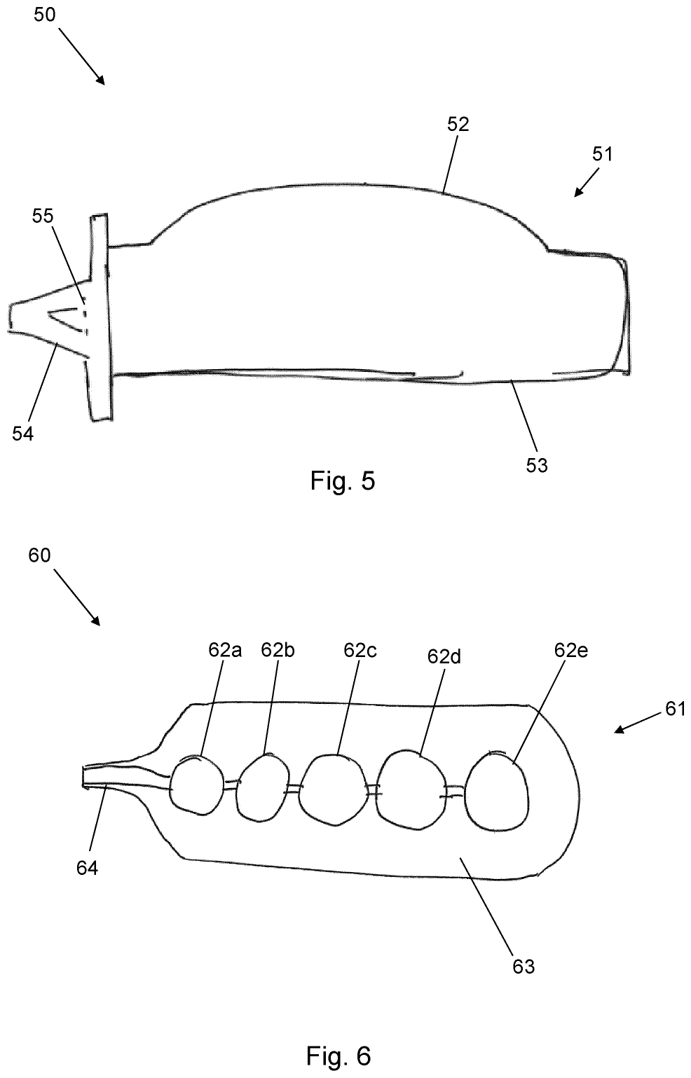

FIG. 5 shows a cross-sectional view of another ampoule 50 according to a further embodiment of the present invention.

The ampoule 50 comprises a body portion 51 and a connector 54 for fitting the ampoule 50 to a cannula or the like. The connector 54 may be a luer connector, such as a luer lock or luer slip connector, or any other suitable connector.

The body portion 51 comprises a flexible portion 52 and a rigid portion 53. In particular, in this embodiment, the rigid portion 53 forms a base and sidewalls of the body portion 51, with the flexible portion 52 providing an upper surface, or at least a portion of the upper surface, of the body portion 51.

As shown in Fig, 5, the flexible portion 52 is configured to bulge outwardly away from the rigid portion 53 when the body portion 51 contains fluid for delivery.

In order to deliver fluid to a patient, a user can compress the body portion 51 by pressing the flexible portion 52 (e.g., downwards or inwards towards the base section formed by the rigid portion 53), which expels fluid out of the ampoule 50 through the connector 54. In particular, in this arrangement, the body portion 51 is preferentially compressible in one plane, which may help to avoid accidentally expelling fluid from the body portion 51 and provide increased control of the fluid flow rate.

In some examples, the flexible portion 52 and the rigid portion 53 may be made of the same material, such as a plastic material or an elastomeric material. For example, the flexible portion 52 may have a reduced thickness (e.g., 0.5 mm) compared with the rigid portion 53 (which may have a thickness or 1.2 mm, for example). In other examples, the flexible portion 52 may comprise a different material from the rigid portion 53. Both the flexible portion 52 and the rigid portion 53 may comprise plastic or elastomeric materials, but it will be appreciated that any other suitable material may also be chosen.

Between the body portion 51 and the connector 54, the ampoule 50 further comprises a valve 55. For example, the valve 55 may be a valve as described below with respect to one of FIGS. 8-11. The valve 55 allows fluid to flow out of the ampoule 50 when the body portion 51 is compressed by a user, but prevents fluid (e.g., air, or liquid) flow back into the ampoule 50 when the compressing force is removed.

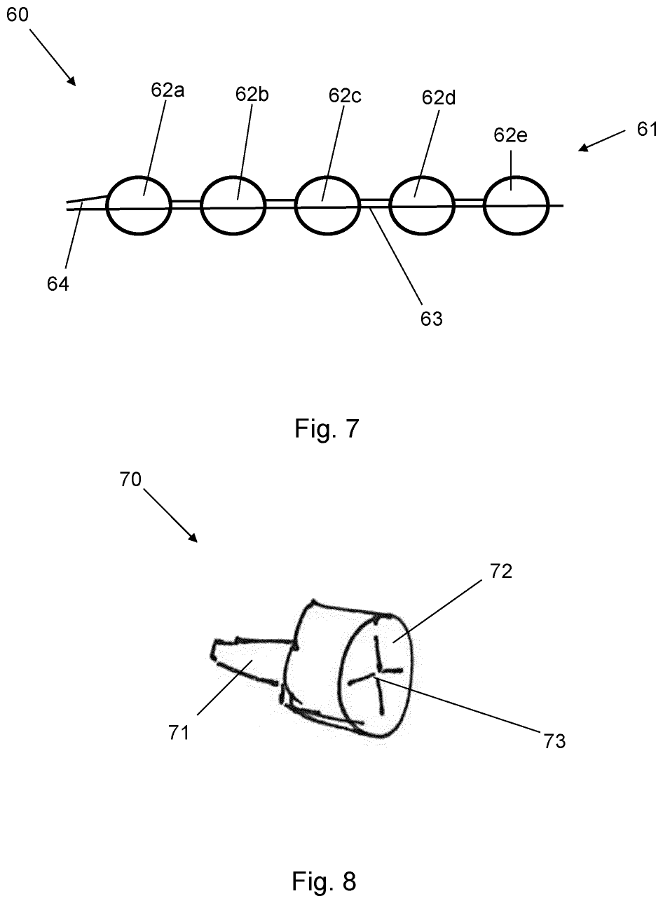

FIG. 6 shows a top view of a further ampoule 60 which is another embodiment of the present invention. FIG. 7 shows a side view of the ampoule 60.

The ampoule comprises a body portion 61 and a connector 64 for fitting the ampoule to a cannula or the like. The connector 64 may be a luer connector, such as a luer lock or luer slip connector, or any other suitable connector.

The body portion 61 comprises five flexible portions 62a-62e and a rigid portion 63 in the form of a cuff or circumferential ridge around the body portion 61. The flexible portions 62a-62e are fluidly connected to one another, and are provided in a similar form to blister capsules, wherein each flexible portion 62a-62e is configured to contain an amount of fluid to be delivered. A user may compress the body portion 61 by applying pressure to one or more of the flexible portions 62a-62e in order to expel fluid from the ampoule 60 through the connector 64. In this way, the ampoule 60 may be used to provide measured unit doses of the fluid (e.g., wherein each flexible portion 62a-62e contains a measured dose of fluid). Furthermore, by being provided in this way, the body portion 61 is preferentially compressible in one plane, which may help to avoid accidentally expelling fluid from the body portion 51 and provide increased control of the fluid flow rate.

It will be appreciated that, in certain embodiments, the ampoule 60 may be further provided with a valve, for example a valve as described below with respect to one of FIGS. 8-11.

FIG. 8 shows a perspective view of a first valve arrangement 70 which may be used in embodiments of the present invention, for example any of the ampoules described above with respect to FIGS. 1-7. In this arrangement, the valve 70 is part of a connector 71 (e.g., a luer connector such as a luer lock or luer slip connector), for example by being integrally formed with the connector 71 or being fixed to the connector 71.

The valve arrangement 70 comprises a sheet of material 71 which covers a first end of the connector 71 (i.e., an end which faces a body portion containing a fluid to be delivered). An opening 73 is formed through the sheet 71, in this embodiment the opening 73 is provided in the form of two slits arranged in the form of a cross or ‘+’ shape. In other examples, the opening 73 may be formed by a single slit, or any number of slits arranged in any suitable manner, or in any other form. By being provided in this way, fluid may pass through the opening 73 in one direction, to flow out of an ampoule through the connector 71, as the slits open to allow fluid to pass through. However, the opening 73 closes when the flow of fluid reverses, preventing fluid (e.g., air, or a liquid) from passing back through the connector 71 and into the body portion of the ampoule.

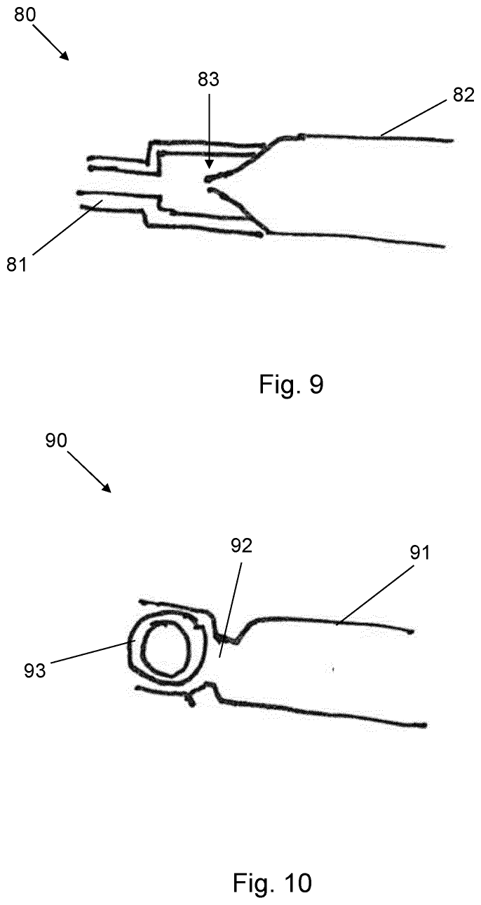

FIG. 9 shows a cross-sectional view through a second valve arrangement 80 which may be used in embodiments of the present invention. In this example, the valve is positioned in a ‘neck’ region of the ampoule between a connector 81 and a body portion 82.

This valve may be referred to as a duckbill valve, and comprises two flaps 83 formed from or connected to an end of the body portion 82. The flaps 83 separate from one another when fluid flows out of the body portion 82 to allow fluid to flow through the connector 82. However, when fluid flows in the reverse direction, the flaps 83 close to prevent fluid flow into the body portion 82.

FIG. 10 shows a cross-sectional view through a third valve arrangement 90 which may be used in embodiments of the present invention. The valve may be positioned in a ‘neck’ region of the ampoule between a connector (not shown) and a body portion 91.

The valve arrangement 90 comprises an opening 92 between the body portion 91 and the connector, and a ball 93 which is configured to sit within the opening 92, on a ‘downstream’ side of the opening, in order to close the opening 92 to fluid flow. When the body portion 91 is compressed, fluid is forced against the ball 93 to unseat the ball from the opening 92 (that is, fluid flow pushes the ball 93 downstream of the opening 92) and thus allow fluid to flow out of the body portion 91 through the connector to be delivered to a patient. However, fluid flow in the reverse direction (i.e., in an ‘upstream’ direction, into the body portion 91) is prevented by the ball's 93 position within the opening 92, as the fluid flow in the reverse direction acts to push the ball 93 so as to seal the opening 92.

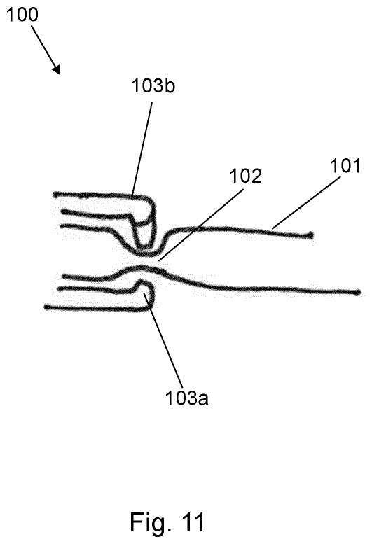

FIG. 11 shows a cross-section view through a fourth valve arrangement 100 which may be used in embodiments of the present invention. In this example, the valve arrangement 100 comprises a section 102 of the body portion 101 which is squeezed or compressed between two jaws 103a, 103b. For example, the jaws 103a, 103b may be part of a connector or may be a separate component or components. The jaws 103a, 103b are generally urged towards one another in order to completely compress the section 102 of the body portion 101 therebetween in order to prevent fluid flow into or out of the body portion 101. For example, the jaws 103a, 103b may be formed of a resilient material such that the jaws 103a, 103b are naturally urged towards one another, or, in some examples, the jaws 103a, 103b may be urged towards one another by one or more biasing elements such as a spring or the like.

When a user compresses the body portion 101, fluid within the body portion presses against the jaws 103a, 103b in order to allow fluid to flow through section 102 and out of the body portion 101 to be delivered to a patient.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention. For example, features of different described embodiments may be combined or altered without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An ampoule for delivering a fluid directly to a patient, the ampoule comprising:
a body portion which is compressible to expel fluid from the ampoule, and
a connector for fitting the ampoule to a cannula or a hollow needle to deliver fluid therethrough.

2. An ampoule according to claim 1, wherein the ampoule further comprises a valve to prevent fluid flow into the ampoule.

3. An ampoule according to claim 1, wherein the body portion is configured to maintain a compressed state after a compressing force has been applied.

4. An ampoule according to claim 1, wherein the body portion comprises:
a rigid portion, and
a flexible portion, wherein the body portion is compressible by applying pressure to the flexible portion.

5. An ampoule according to claim 4, wherein either:
the rigid portion and the flexible portion comprise the same material, or the rigid portion and the flexible portion comprise different materials.

6. An ampoule according to claim 1, wherein the body portion is preferentially compressible in one dimension.

7. An ampoule according to claim 6, wherein the body portion is generally tubular with an elliptical cross-section.

8. An ampoule according to claim 1, further comprising a circumferential ridge around the body portion for stiffening the body portion.

9. An ampoule according to claim 8, wherein the circumferential ridge is a planar flange which extends outwardly from the ampoule.

10. An ampoule according to claim 8, wherein either:
the circumferential ridge is formed of a different material to the body portion, or
the circumferential ridge is formed of the same material as the body portion.

11. An ampoule according to claim 1, wherein the body portion comprises a plastic material, and/or an elastomeric material.

12. An ampoule according to claim 1, wherein the ampoule tapers towards a first end and wherein liquid is expelled from the ampoule through the first end.

13. An ampoule according to claim 1, further comprising a tamper-evident closure.

14. An ampoule according to claim 1, wherein the connector is either:
a luer slip fitting, or
a luer lock fitting.

15. An ampoule according to claim 1, wherein the ampoule is made of a single material.

16. An ampoule according to claim 1, wherein the fluid is an injectable medicament.

17. An apparatus for delivering fluid to a patient, the apparatus comprising:
an ampoule according to claim 1; and
a cannula or a catheter.

18. An apparatus for delivering fluid to a patient, the apparatus comprising:
an ampoule according to claim 1; and
a hollow needle.
